(19) United States
(12) Patent Application Publication
(10) Pub. No.: US 2015/0173686 A1
(43) Pub. Date: Jun. 25, 2015

(54) BIOLOGICAL INFORMATION MEASURING DEVICE AND CONTROL METHOD FOR BIOLOGICAL INFORMATION MEASURING DEVICE

(71) Applicant: Seiko Epson Corporation, Shinjuku-ku (JP)

(72) Inventors: Naoshi FURUTA, Shiojiri-shi (JP); Shintaro Nagasaki, Hara-Mura (JP)

(21) Appl. No.: 14/579,993

(22) Filed: Dec. 22, 2014

(30) Foreign Application Priority Data
Dec. 25, 2013 (JP) 2013-266634

(51) Int. Cl. A61B 5/00; A61B 5/11; A61B 5/0205

(52) U.S. Cl. CPC A61B 5/7285; A61B 5/02055; A61B 5/1123; A61B 5/0002; A61B 5/681

(57) ABSTRACT

A biological information measuring device includes a biological-information detecting unit configured to detect biological information of a human body on which the biological information measuring device is worn, an acceleration detecting unit configured to detect acceleration, a mode setting unit configured to set, among a plurality of operation modes set on the basis of a change in the detected acceleration, an operation mode corresponding to a change pattern of the acceleration, and an executing unit configured to execute processing corresponding to the set operation mode.

This application claims priority to Japanese Patent Application No. 2013-266634, filed Dec. 25, 2013, the entirety of which is hereby incorporated by reference.

### BACKGROUND

1. Technical Field

The present invention relates to a biological information measuring device and a control method for the biological information measuring device.

2. Related Art

As a biological information measuring device that measures biological information, there is know a measuring device configured in a form of a bracelet (a wristwatch), which is worn on a wrist, and configured to measure a pulse rate of a user (see, for example, JP-A-2006-312010 (Patent Literature 1)).

The measuring device (a sensor node) described in Patent Literature 1 includes a square case to which a band is attached, a display device, a pulse sensor, a temperature sensor, and an acceleration sensor. A measurement switch for causing the measuring device to execute processing for measuring biological information is provided on the surface of the case. The measuring device transmits data such as temperature and a pulse sensed by the sensors to a base station through radio communication. The base station communicates with a management server present in a remote place via a wide area network. The management server manages the data collected from the base station using a database.

Incidentally, it is desired that a wearable device worn on a human body (e.g., a wrist) and used is small in size and light in weight in order to reduce a burden on a user.

As in the measuring device described in Patent Literature 1, if buttons (switches) for operating the measuring device are protrudingly provided on the case, the measuring device tends to be increased in size. Besides, design flexibility decreases. On the other hand, it is conceivable to provide a touch panel for operating the measuring device on the case. However, it is necessary to secure an operation area of the touch panel relatively large in order to allow the user to easily operate the touch panel. Therefore, the measuring device tends to be increased in size.

### SUMMARY

An advantage of some aspects of the invention is to provide a biological information measuring device and a control method for the biological information measuring device that can reduce a burden on a user.

A biological information measuring device according to a first aspect of the invention includes: a biological-information detecting unit configured to detect biological information of a human body on which the biological information measuring device is worn; an acceleration detecting unit configured to detect acceleration; a mode setting unit configured to set, among a plurality of operation modes set on the basis of a change in the detected acceleration, an operation mode corresponding to a change pattern of the acceleration; and an executing unit configured to execute processing corresponding to the set operation mode.

In the following explanation, the “biological information measuring device” is sometimes simply referred to as “measuring device”.

Note that example of the biological information include a pulse wave, a body temperature, a blood pressure, an electrocardiogram, and a brain wave.

According to the first aspect, by moving the measuring device or applying a predetermined shock to the measuring device, it is possible to change the operation mode of the measuring device to another operation mode and easily cause the measuring device to execute processing corresponding to the set operation mode. Therefore, the processing to be executed can be changed according to the change pattern of the acceleration without depending on input operation of a button or the like. Therefore, it is possible to attain a reduction in the size of the measuring device and reduce a burden on a user. On the other hand, even when the same operation (e.g., continuous tap operation) is carried out when different operation modes are set, it is possible to execute processing corresponding to the set operation modes. Further, since the processing can be executed without providing a machine mechanism such as a button exposed to the outside, it is possible to secure a waterproof property without providing a special component. Consequently, it is also possible to reduce costs.

In the first aspect, it is preferable that, when the operation mode is a standby mode for standing by for detection of the biological information by the biological-information detecting unit, if the acceleration changes, the mode setting unit switches the operation mode to a detection mode for detecting the biological information.

Note that the acceleration changes when the operation mode is the standby mode, for example, if the measuring device left untouched on a table is lifted by the user.

The measuring device in the past is often configured to start detection and measurement of biological information when measurement start operation such as a button input or the like is carried out like an input of the measurement switch. In such a configuration, when the measurement start operation is forgotten, although the measuring device is worn, the detection and the measurement of the biological information are not carried out.

On the other hand, according to the first aspect with the configuration described above, when the acceleration changes in the standby mode, for example, when the measuring device left untouched on the table is lifted to be worn on the human body, the operation mode is switched from the standby mode to the detection mode by the mode setting unit. Consequently, it is possible to carry out the detection of the biological information when the measuring device is worn and surely carry out measurement of the biological information. Therefore, it is possible to suppress occurrence of a measurement omission of the biological information.

In the first aspect, it is preferable that, when the operation mode is a standby mode for standing by for detection of the biological information by the biological-information detecting unit, if a change in the acceleration corresponding to a continuous shock is detected, the mode setting unit switches the operation mode to a power saving mode for reducing power consumption to be smaller than power consumption in the standby mode.

Note that examples of the shock include a shock by tap operation for tapping the measuring device with a hand, a finger, or the like.

A wearable device worn on a human body is generally configured to operate with electric power supplied from a primary battery or a secondary battery. However, if power consumption of the device is large, the battery needs to be frequently replaced or charged. Convenience of the device is deteriorated.

On the other hand, in the first aspect with the configuration described above, when the change in the acceleration corresponding to the continuous shock is detected, the operation mode is switched from the standby mode to the power saving mode by the mode setting unit. Consequently, it is possible to reduce power consumption of the measuring device. For example, when biological information is not measured, by continuously performing the tap operation on the measuring device, it is possible to switch the operation mode to the power saving mode and reduce the power consumption of the measuring device. Therefore, it is unnecessary to frequency replace or charge the battery. It is possible to improve convenience of the measuring device.

When the operation mode is switched to the power saving mode, the change in the acceleration corresponding to the continuous shock needs to occur. Therefore, when some shock acts on the measuring device only once, the operation mode is not switched. Therefore, it is possible to suppress the operation mode from being frequently switched to the power saving mode.

In the first aspect, it is preferable that the measuring device further includes a regulating unit configured to regulate, when the operation mode is a measurement mode for measuring the biological information, setting of the power saving mode by the mode setting unit.

When the operation mode is the measurement mode, if the change in the acceleration is detected and the operation mode is switched to the power saving mode, it is likely that the operation mode is switched to the power saving mode before the measurement of the biological information is completed. In such a case, it is likely that the biological information cannot be appropriately measured.

On the other hand, according to the first aspect with the configuration described above, since the regulating unit regulates the switching from the measurement mode to the power saving mode, it is possible to appropriately measure the biological information.

In the first aspect, it is preferable that, when the operation mode is the power saving mode, if the change in the acceleration corresponding to the continuous shock is detected, the mode setting unit releases the power saving mode.

According to the first aspect with the configuration described above, it is possible to easily switch the operation mode from the power saving mode to an operation mode in which the measuring device can be used (e.g., the standby mode) and easily execute the detection and the measurement of the biological information. Therefore, it is possible to improve operability of the measuring device and suppress occurrence of a measurement omission of the biological information.

In the first aspect, it is preferable that the measuring device further includes a communication unit configured to communicate with an external apparatus, and, when the operation mode is the power saving mode, if electric power is supplied from the outside, the mode setting unit switches the operation mode to a communication mode in which the measuring device can communicate with the external apparatus via the communication unit.

According to the first aspect with the configuration described above, the operation mode is switched from the power saving mode to the communication mode according to the power supply from the outside. Power consumption of the communication with the external apparatus is relatively large. However, since the operation mode is switched to the communication mode according to the power supply, it is possible to perform the communication using the supplied power. Therefore, it is possible to communicate with the external apparatus without the communication connection being interrupted by battery exhaustion.

In the first aspect, it is preferable that, when the electric power is supplied from the outside, if the change in the acceleration corresponding to the continuous shock is detected, the mode setting unit switches the operation mode to a communication release mode for releasing the communication with the external apparatus, and the executing unit deletes connection information with the external apparatus stored in advance.

According to the first aspect with the configuration described above, when the operation mode is switched to the communication release mode by the mode setting unit, since the connection information is deleted by the executing unit, it is possible to release the communication with the external apparatus associated with the measuring device. Therefore, since it is possible to connect the measuring device to a different external apparatus and change a connection destination of the measuring device, it is possible to improve the convenience of the measuring device.

In the first aspect, it is preferable that, when the power supply from the outside is stopped, after switching the operation mode to a reset mode and causing the executing unit to reset the biological information measuring device, the mode setting unit switches the operation mode to a start mode for starting the biological information measuring device.

An electronic device is desirably reset at every predetermined period to prevent the operation of the electronic device from becoming unstable. However, in a wearable device worn on a human body and used for a long time, it is difficult to obtain timing of the reset.

On the other hand, in the first aspect with the configuration described above, the operation mode is switched to the start mode through the reset mode at timing when the power supply from the outside is stopped, that is, timing when the measuring device is not worn on the human body. Consequently, it is possible to execute the reset processing without the user being aware of the reset processing and cause the measuring device to stably operate. Start processing after the reset requires relatively large electric power. However, when the measuring device includes a secondary battery charged by supplied electric power, it is assumed that electric power for executing the start processing is stored by the secondary battery. Therefore, it is possible to suppress the start processing from being stopped by battery exhaustion.

A control method for a biological information measuring device according to a second aspect of the invention is a control method for a biological information measuring device worn on a human body to measure biological information, the control method including: detecting acceleration; setting, among a plurality of operation modes set on the basis of a change in the detected acceleration, an operation mode corresponding to a change pattern of the acceleration; and executing processing corresponding to the set operation mode.

According to the second aspect, by applying the control method to the biological information measuring device, it is possible to attain effects same as the effects of the biological measuring device according to the first aspect.

A biological information measuring device according to a third aspect of the invention includes: a biological-information detecting unit configured to detect biological information of a human body on which the biological information measuring device is worn; an acceleration detecting unit configured to detect acceleration; a mode setting unit configured to set an operation mode on the basis of a change in the detected acceleration; and an executing unit configured to execute predetermined processing when a predetermined acceleration change is detected by the acceleration detecting unit in the set operation mode.

With the biological information measuring device according to the third aspect, it is possible to attain effects same as the effects of the biological information measuring device according to the first aspect.

### BRIEF DESCRIPTION OF THE DRAWINGS

The invention will be described with reference to the accompanying drawings, wherein like numbers reference like elements.

FIG. 1 is a plan view showing a biological information measuring device according to an embodiment of the invention.

FIG. 2 is a perspective view showing the biological information measuring device in the embodiment.

FIG. 3 is a block diagram showing the configuration of the biological information measuring device in the embodiment.

FIG. 4 is a plan view showing a display unit in the embodiment.

FIG. 5 is a block diagram showing the configuration of a control unit in the embodiment.

FIG. 6 is a transition diagram showing an operation mode of the biological information measuring device in the embodiment.

FIG. 7 is a diagram showing the display unit in a trial mode in the embodiment.

FIG. 8 is a diagram showing a display state by the display unit corresponding to the number of times of taps in the embodiment.

FIG. 9 is a diagram showing a display state by the display unit during transition to a power saving mode in the embodiment.

FIG. 10 is a diagram showing a display state by the display unit in a communication release mode in the embodiment.

FIG. 11 is a diagram showing a display state by the display unit in an update mode in the embodiment.

FIG. 12 is a flowchart for explaining first mode setting processing in the embodiment.

FIG. 13 is a flowchart for explaining second mode setting processing in the embodiment.

FIG. 14 is a flowchart for explaining third mode setting processing in the embodiment.

### DESCRIPTION OF EXEMPLARY EMBODIMENTS

### External Configuration of a Biological Information Measuring Device.

An embodiment of the invention is explained below with reference to the drawings.

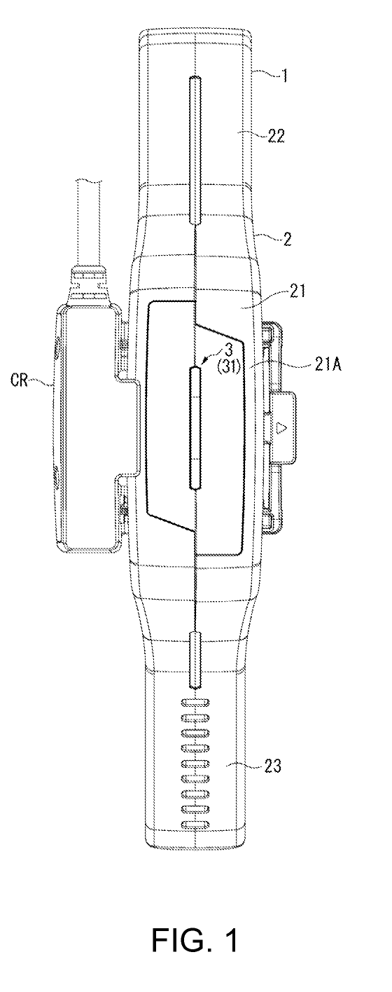

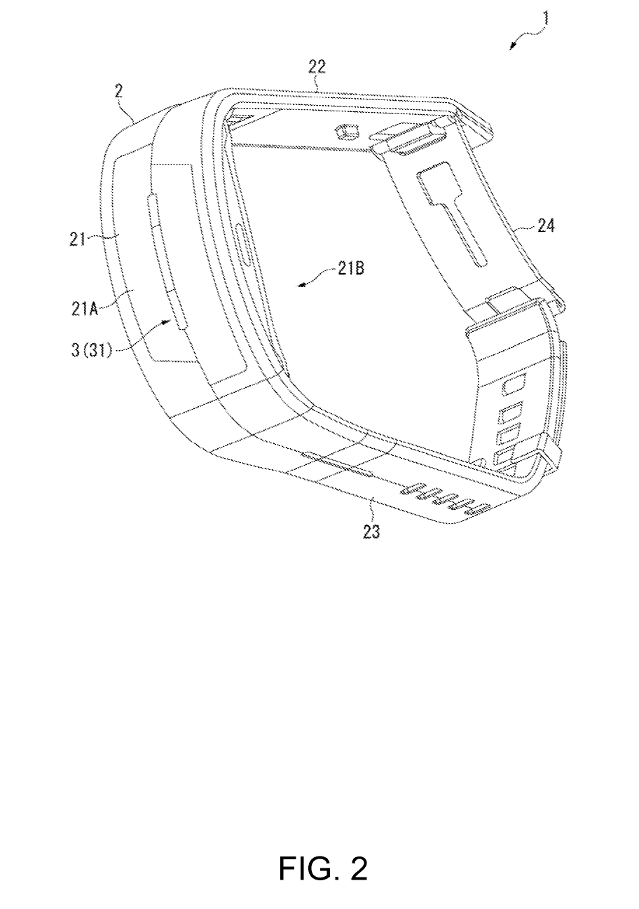

FIG. 1 is a diagram showing a state in which a biological information measuring device 1 according to this embodiment is connected to a cradle CR. FIG. 2 is a perspective view showing the biological information measuring device 1.

As shown in FIG. 1, the biological information measuring device (hereinafter sometimes abbreviated as measuring device) 1 according to this embodiment is a device that is, after being connected to the cradle CR for power supply and charged, worn on the wrist of a user and detects a pulse wave and measures a pulse rate as biological information. That is, the measuring device 1 is a wearable device wearable on a human body.

As shown in FIGS. 1 and 2, the measuring device 1 includes a case 2 that configures an armor. The case 2 includes a main body unit 21 and a pair of bands 22 and 23 respectively integrally configured and a buckle 24 (FIG. 2) attached to the bands 22 and 23.

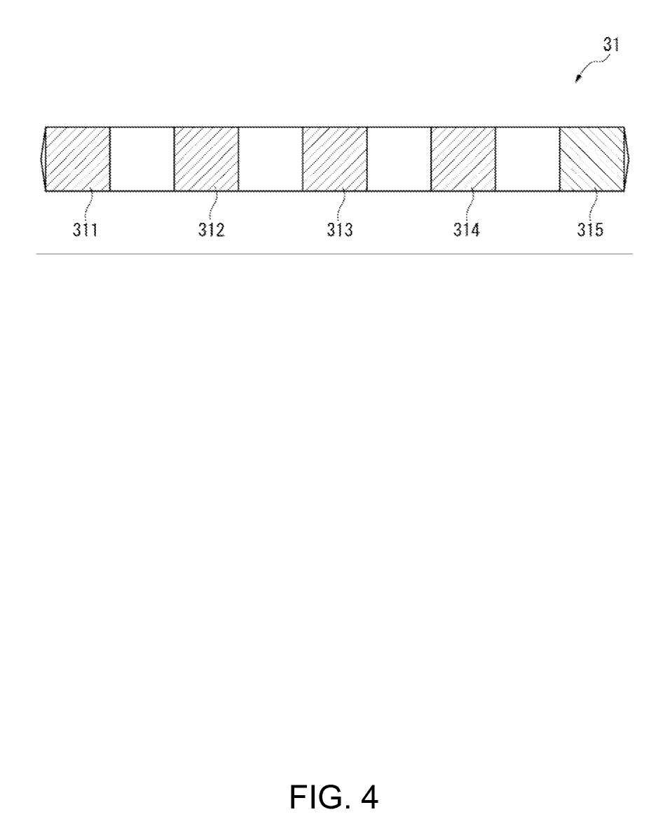

As shown in FIG. 2, the main body unit 21 is configured in a substantially arcuate shape in side view corresponding to an outer side part of the wrist (a part on the side of the back of the hand) on which the measuring device 1 is worn. In a front surface section 21A of the main body unit 21, as shown in FIGS. 1 and 2, a display unit 31 of a notifying unit 3 explained below is provided. Although not shown in FIGS. 1 and 2, a biological-information detecting unit 41 (see FIG. 4) of a detecting unit 4 explained below is exposed to a rear surface section 21B on the opposite side of the front surface section 21A.

The pair of bands 22 and 23 extends in opposite directions each other from one end and the other end in the longitudinal direction of the main body unit 21.

The buckle 24 is a clasp called D buckle that fixes the bands 22 and 23 when the measuring device 1 is worn on the wrist.

In this way, it is one of objects of the measuring device 1 to attain a reduction in the size and improvement of a design property of the measuring device 1. The measuring device 1 is based on the premise that the measuring device 1 is operated by tap operation (operation for tapping the measuring device 1 with a hand, a finger, or the like) of the user. An operation unit such as a button is not provided on the case 2. However, the measuring device 1 is not limited to this. An operation unit such as buttons or a touch panel for causing the measuring device 1 to execute predetermined processing may be provided on the case 2.

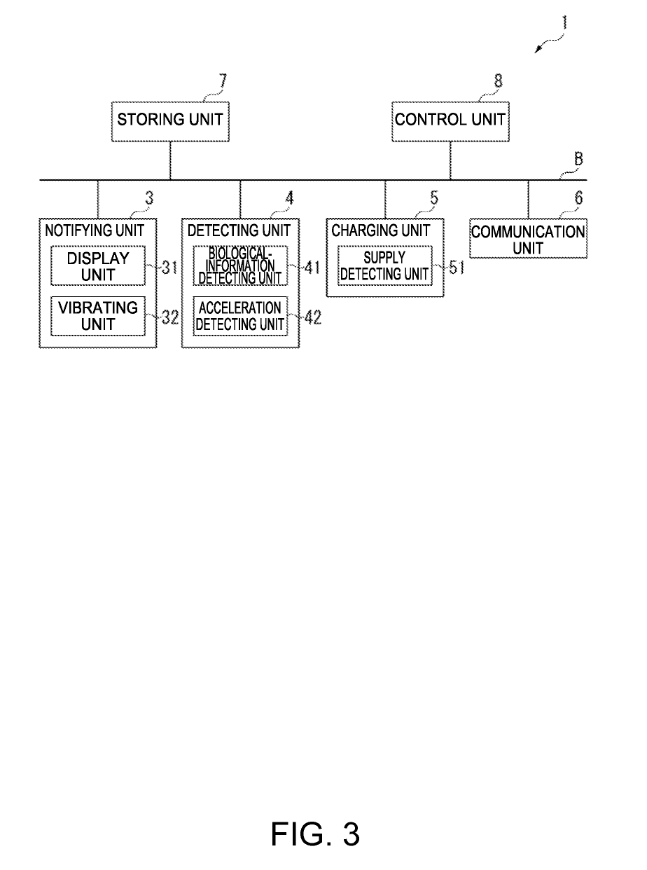

FIG. 3 is a block diagram showing the configuration of the measuring device 1.

The measuring device 1 includes, besides the case 2, as shown in FIG. 3, a notifying unit 3, a detecting unit 4, a charging unit 5, a communication unit 6, a storing unit 7, a control unit 8, and a secondary battery (not shown in the figure) that supplies electric power to the components of the measuring device 1. The units 3 to 8 are electrically connected by a bus B.

### Configuration of the Notifying Unit.

The notifying unit 3 notifies an operation state and an operating state of the measuring device 1. The notifying unit 3 includes a display unit 31 and a vibrating unit 32.

The vibrating unit 32 includes a motor controlled by a notification control unit 82. The vibrating unit 32 notifies a state of the measuring device 1 using vibration caused by driving of the motor.

### Configuration of the Display Unit.

FIG. 4 is a plan view showing the display unit 31.

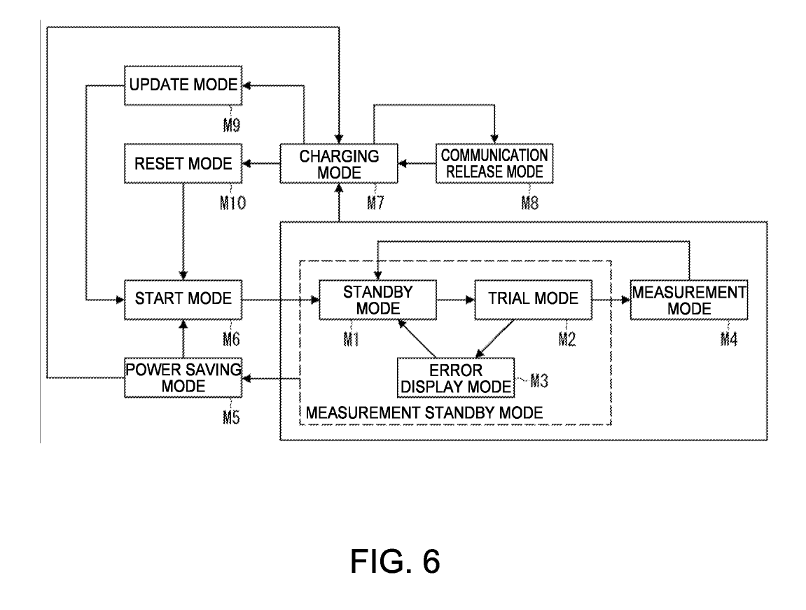

The display unit 31 includes, as shown in FIG. 4, five LEDs (Light Emitting Diodes) 311 to 315 arrayed along the longitudinal direction of the main body unit 21. Under the control by the notification control unit 82, the display unit 31 displays a state of the measuring device 1 according to lighting (including blinking) and extinction of the LEDs 311 to 315. For example, the display unit 31 switches, every time the tap operation is performed, the LEDs from an extinguished state to a lit state one by one to display the number of times of the tap operation (hereinafter sometimes referred to as the number of times of taps). When the operation mode is switched to a start mode M6 (see FIG. 6) explained below, the display unit 31 switches an LED to be lit among the LEDs 311 to 315 in order from the LED 311 to indicate that the measuring device 1 is performing start processing.

In this embodiment, among the LEDs 311 to 315, LEDs having different colors during light emission are respectively adopted as four LEDs 311 to 314 located on one end side (the lower side in FIG. 1) and one LED 315 located on the other end side (the upper side in FIG. 1). Specifically, LEDs that emit blue light are adopted as the LEDs 311 to 314. An LED that emits orange light is adopted as the LED 315. This makes it easy to grasp whether the number of times of the tap operation by the user reaches the number of times of taps for switching to a certain operation mode (the number of times of taps for causing the measuring device 1 to execute a certain function; in this embodiment, five times). However, the LEDs 311 to 315 are not limited to this. All of the LEDs 311 to 315 may have the same color during light emission. An arrayed direction of the LEDs 311 to 315 is not limited to the longitudinal direction. For example, the LEDs 311 to 315 may be arrayed obliquely to the front surface section 21A of the main body unit 21.

### Configuration of the Detecting Unit.

The detecting unit 4 includes a biological-information detecting unit 41 and an acceleration detecting unit 42 respectively functioning as the biological-information detecting unit and the acceleration detecting unit according to the invention.

The biological information detecting unit 41 detects biological information of the user who wears the measuring device 1. In this embodiment, the biological-information detecting unit 41 is a pulse-wave detecting unit that detects a pulse wave serving as biological information. The biological-information detecting unit 41 is exposed to the rear surface section 21B (a part opposed to the human body) of the case 2.

The biological-information detecting unit 41 includes a photoelectric sensor including a light emitting element such as an LED and a light receiving element such as a photodiode. The photoelectric sensor causes the light emitting element to irradiate light on a living organism in a state in which the measuring device 1 is worn on the wrist. The photoelectric sensor detects a light amount change in receiving, with the light receiving element, light arriving through a blood vessel of the living organism to detect a pulse wave. The light irradiated on the living organism is partially absorbed by the blood vessel. However, an absorption ratio in the blood vessel changes according to the influence of pulsation. An amount of light reaching the light receiving element changes. An executing unit 92 explained below can measure a pulse rate (a pulse rate per unit time) by analyzing a temporal change, that is, a pulse wave of the light amount detected by the light receiving element.

When the measuring device 1 is detached from the wrist and changes to an unworn state, the light receiving element detects external light. The external light is extremely strong light compared with reflected light and transmitted light of the light from the light receiving element. That is, when the measuring device 1 is in the unworn state, the light amount detected by the light receiving element is extremely large compared with a light amount detected when the measuring device 1 is in the worn state. Therefore, by determining a light amount detected by the light receiving element, it is possible to determine whether the measuring device 1 is worn on the human body.

Note that, in this embodiment, the biological-information detecting unit 41 functioning as the pulse-wave detecting unit includes the photoelectric sensor. However, for example, the biological-information detecting unit 41 may include an ultrasonic sensor that detects contraction of a blood vessel with ultrasound and measures a pulse rate. The biological-information detecting unit 41 may include a sensor, a piezoelectric element, or the like that feeds a feeble current from an electrode into a body and detects a pulse.

The acceleration detecting unit 42 includes an acceleration sensor that detects an acceleration value involved in a motion of the user wearing the measuring device 1. The acceleration detecting unit 42 outputs the detected acceleration value to the control unit 8. Examples of the acceleration sensor include a three-axis sensor that detects, at a predetermined sampling frequency, acceleration values in respective axes of an X axis, a Y axis, and a Z axis.

A change pattern of an acceleration value detected when a motion for swinging the wrist or a motion for rotating the wrist is performed by the user in a state in which the measuring device 1 is worn on the wrist and a change pattern of an acceleration value detected when tap operation is performed on the measuring device 1 are different. Therefore, by determining the patterns, it is possible to determine whether the tap operation is performed on the measuring device 1.

Note that the acceleration detecting unit 42 may include a tap operation detecting function for determining whether the tap operation is carried out by the acceleration detecting unit 42 itself and, when determining that the tap operation is performed, outputting an interruption signal to the control unit 8. The acceleration value detected by the acceleration detecting unit 42 can also be used for processing for reducing noise due to a body motion superimposed on a pulse wave signal detected by the biological-information detecting unit 41 functioning as the pulse-wave detecting unit.

### Configuration of the Charging Unit.

The charging unit 5 is configured by a charging circuit. The charging unit 5 charges the secondary battery with electric power supplied from the cradle CR via a terminal (not shown in the figure) exposed to the outside of the case 2. The charging unit 5 includes a supply detecting unit 51 that detects a voltage supplied to the terminal. The charging unit 5 outputs, to the control unit 8, a notification signal for notifying presence or absence of power supply from the outside. Therefore, the control unit 8 can determine on the basis of the notification signal whether power supply to the measuring device 1 is performed. That is, the control unit 8 can determine whether the measuring device 1 and the cradle CR are connected.

### Configuration of the Communication Unit.

The communication unit 6 is a module that communicates with an external apparatus under the control by a communication control unit 83 explained below. In this embodiment, the communication unit 6 is configured by a module that communicates with the external apparatus by radio (i.e., a module conforming to a short-range radio communication standard such as an IEEE802.15 standard). The communication unit 6 may be a module that communicates with the external apparatus by wire. Further, the communication unit 6 may be a module capable of communicating with the external apparatus by radio and by wire.

### Configuration of the Storing Unit.

The storing unit 7 has stored therein a computer program and data necessary for the operation of the measuring device 1. As the computer program, the storing unit 7 has stored therein a computer program for causing the control unit 8 to execute mode setting processing explained below. As the data, the storing unit 7 has stored therein connection information for performing communication connection to the external apparatus via the communication unit 6. Further, the storing unit 7 stores a detection result by the detecting unit 4 under the control by the control unit 8. The storing unit 7 can be configured by a nonvolatile semiconductor memory such as a flash memory.

### Configuration of the Control Unit.

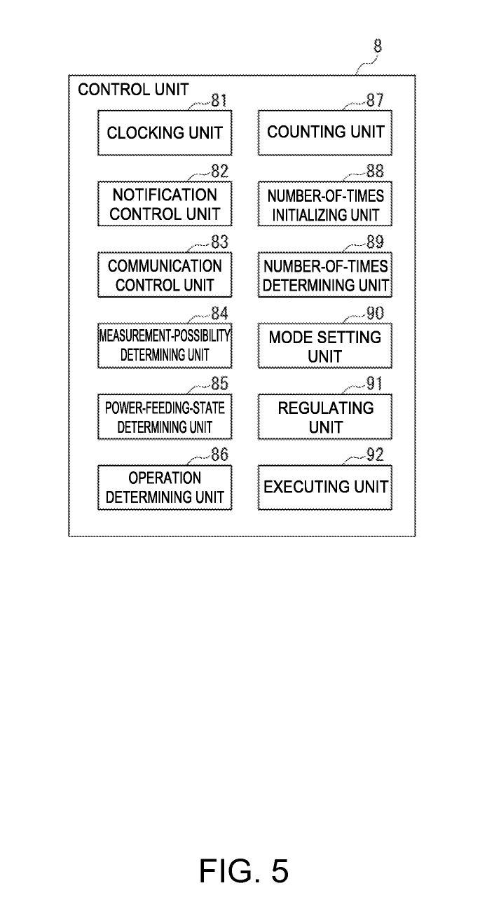

FIG. 5 is a block diagram showing the configuration of the control unit 8.

The control unit 8 is configured by a control circuit and controls the operation of the measuring device 1. That is, the control unit 8 autonomously controls the operation of the measuring device 1. Besides, the control unit 8 changes an operation mode according to operation on the measuring device 1 and a state of the measuring device 1 and executes processing corresponding to the operation mode.

The control circuit executes the computer program stored in the storing unit 7, whereby the control unit 8 includes, as shown in FIG. 5, functional units functioning as a clocking unit 81, the notification control unit 82, a communication control unit 83, a measurement-possibility determining unit 84, a power-feeding-state determining unit 85, an operation determining unit 86, a counting unit 87, a number-of-times initializing unit 88, a number-of-times determining unit 89, a mode setting unit 90, a regulating unit 91, and an executing unit 92.

The clocking unit 81 clocks the present date and time.

The notification control unit 82 controls the display unit 31 and displays an operation state of the measuring device 1, the number of times of taps counted by the counting unit 87, and the like. The notification control unit 82 controls the vibrating unit 32 to generate the vibration.

When the operation mode of the measuring device 1 is a mode in which the measuring device 1 is capable of communicating with the external apparatus, the communication control unit 83 communicates with the external apparatus via the communication unit 6. In the communication, the communication control unit 83 communicates with, on the basis of the connection information stored in the storing unit 7, the external apparatus associated with the measuring device 1.

The measurement-possibility determining unit 84 determines on the basis of a detection result by the biological-information detecting unit 41 whether detection of biological information is possible.

The power-feeding-state determining unit 85 determines on the basis of a notification signal input from the charging unit 5 whether electric power is supplied from the cradle CR, in other words, whether the measuring device 1 and the cradle CR are connected.

The operation determining unit 86 determines a change pattern of acceleration detected by the acceleration detecting unit 42 to thereby determine operation content for the measuring device 1. For example, the operation determining unit 86 executes first determination processing for determining whether an acceleration value changes after being fixed for a predetermined period to determine whether the measuring device 1 placed on a table or the like is lifted. Note that “fixed” does not have to mean “completely fixed”. When the change in then acceleration value is within a predetermined error range, the operation determining unit 86 can determine that the acceleration is fixed.

The operation determining unit 86 executes second determination processing for determining whether a change in an acceleration value corresponding to a continuous shock due to tap operation on the measuring device 1 occurs or whether the interruption signal involved in the tap operation is input to determine whether the tap operation is performed.

When the operation determining unit 86 determines that a change pattern of an acceleration value corresponding to the tap operation is detected (the tap operation is performed), the counting unit 87 counts the number of times of the tap operation (the number of times of taps) and causes the storing unit 7 to store the number of times of taps.

When a predetermined time elapses after the operation determining unit 86 determines last that the tap operation is performed, the number-of-times initializing unit 88 initializes the number of times of taps counted by the counting unit 87. Besides, when the number of times of taps reaches a predetermined number of times (in this embodiment, five times), the number-of-times initializing unit 88 also initializes the number of times of taps.

The number-of-times determining unit 89 determines whether the number of times of taps counted by the counting unit 87 reaches the predetermined number of times.

The mode setting unit 90, the regulating unit 91, and the executing unit 92 are respectively equivalent to the mode setting unit, the regulating unit, and the executing unit according to the invention.

The mode setting unit 90 sets a present operation mode of the measuring device 1 on the basis of determination results by the measurement-possibility determining unit 84, the power-feeding-state determining unit 85, the operation determining unit 86, and the number-of-times determining unit 89. The operation mode set by the mode setting unit 90 is explained in detail below.

The regulating unit 91 regulates the switching of the operation mode by the mode setting unit 90 under a certain condition.

The executing unit 92 executes processing corresponding to the present operation mode set by the mode setting unit 90.

### Transition of the Operation Mode and Processing in Operation Modes.

FIG. 6 is a diagram showing transition of the operation mode of the measuring device 1.

The operation mode of the measuring device 1 and transition of the operation mode are explained below with reference to FIG. 6.

### Explanation of a Standby Mode.

A standby mode M1 is an operation mode for standing by for detection and measurement of biological information by the biological-information detecting unit 41. The standby mode M1 is an operation mode set when the measuring device 1 is not worm on the human body and it is assumed that there is no motion in the measuring device 1 (when a detected acceleration value is fixed for a predetermined period). In the standby mode M1 in this embodiment, a communication function of the communication unit 6 and a detection function of the biological-information detecting unit 41 are disabled. However, the detection function for a motion of the measuring device 1 and the tap operation of the measuring device 1 by the acceleration detecting unit 42 is effective. That is, in the standby mode M1, the first determination processing and the second determination processing by the operation determining unit 86 are executed.

### Explanation of a Trial Mode.

When the operation mode is the standby mode M1, if the operation determining unit 86 determines that there is a change in the acceleration, the mode setting unit 90 switches the present operation mode to a trial mode M2 on the basis of the change pattern of the acceleration.

The trial mode M2 is a mode for trying detection of biological information by the biological-information detecting unit 41 and is equivalent to the detection mode according to the invention. In the trial mode M2 in this embodiment, although the communication function of the communication unit 6 is disabled, the detection function by the detecting units 41 and 42 are effective. In the trial mode M2 in this embodiment, the biological-information detecting unit 41 executes detection of a pulse wave under the control by the executing unit 92. The measurement-possibility determining unit 84 determines whether measurement of a pulse based on the pulse wave can be carried out.

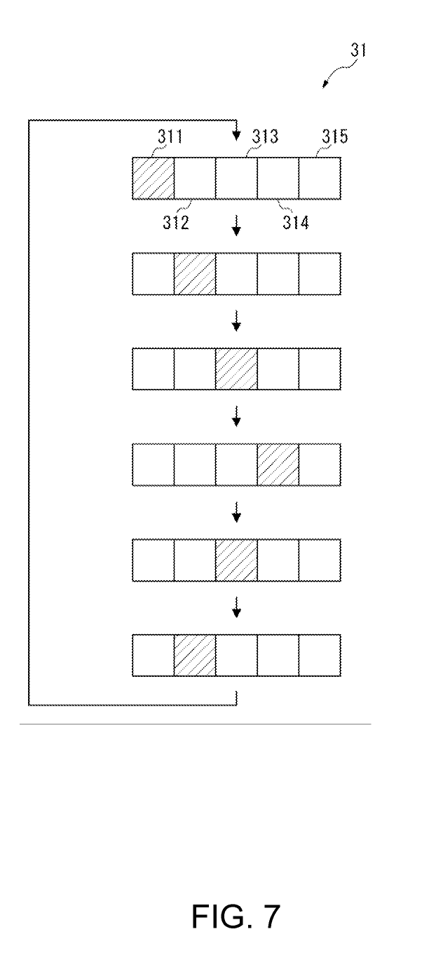

FIG. 7 is a diagram showing the display unit 31 in the trial mode M2 of the operation mode.

Note that, when the present operation mode is the trial mode M2, that is, while the determination processing by the measurement-possibility determining unit 84 is executed, as shown in FIG. 7, the notification control unit 82 controls a lit state of the LEDs 311 to 314, which emit blue light, such that lighting and extinction are sequentially switched from one side to the other side or from the other side to one side in the display unit 31. In this case, in this embodiment, the LED 315 that emits orange light is kept extinguished. However, all the LEDs 311 to 315 may be sequentially lit.

### Explanation of an Error Display Mode.

When the pulse wave cannot be detected even if the operation mode is. switched to the trial mode M2 and a trial time (in this embodiment, five minutes) elapses after detection determination processing for a pulse wave is executed, that is, when the measurement-possibility determining unit 84 determines that detection of biological information is impossible, as shown in FIG. 6, the mode setting unit 90 switches the present operation mode to an error display mode M3.

The error display mode M3 is an operation mode for indicating that a pulse wave cannot be detected. In the error display mode M3 in this embodiment, as in the standby mode M1, whereas the communication function of the communication unit 6 and the detection function of the biological-information detecting unit 41 are disabled, the detection function of the acceleration detecting unit 42 is effective.

In the error display mode M3, although not shown in the figure, when a pulse wave cannot be detected because, for example, the measuring device 1 is not correctly worn on the human body, the notification control unit 82 turns on and off the LEDs 311 and 315 for a predetermined time (e.g., ten seconds) under the control by the executing unit 92. When a pulse wave cannot be detected because of low temperature, similarly, the notification control unit 82 turns on and off the LEDs 314 and 315. Further, when the operation mode is switched to the error display mode M3, the notification control unit 82 drives the vibrating unit 32 for a predetermined time (e.g., three seconds) under the control by the executing unit 92. Occurrence of a detection error is notified to the user by generated vibration.

### Explanation of a Measurement Mode.

On the other hand, when a pulse wave is detected within the trial time and the measurement-possibility determining unit 84 determines that measurement of biological information is possible, the mode setting unit 90 switches the present operation mode to a measurement mode M4.

The measurement mode M4 is an operation mode in which the executing unit 92 analyzes a pulse wave detected by the biological-information detecting unit 41, measures a pulse (a pulse rate per unit time), and stores the pulse in the storing unit 7. In the measurement mode M4 in this embodiment, the communication function of the communication unit 6 and the detection function of the detecting units 41 and 42 are effective. Therefore, the executing unit 92 can transmit a measurement result to the external apparatus on a real time basis.

Although not shown in the figure, when the operation mode is transitioned to the measurement mode M4, the notification control unit 82 turns on and off all of the LEDs 311 to 315 a predetermined number of times (e.g., twice) under the control by the executing unit 92. Consequently, the start of measurement of a pulse rate is notified to the user.

Note that, after the operation mode is switched to the measurement mode M4, when the measuring device 1 is in the unworn state for a predetermined time (e.g., three minutes) (i.e., when biological information is not detected or when there is no change in a detected acceleration value), the mode setting unit 90 switches the operation mode to the standby mode M1. Such a worn state of the measuring device 1 is determined on the basis of the intensity of light received by the light receiving element of the biological-information detecting unit 41 and a change in an acceleration value as explained above.

### Explanation of a Power Saving Mode.

When the operation mode is a measurement standby mode including the modes M1 to M3, if the operation determining unit 86 determines that a change in acceleration corresponding to a shock (a change in acceleration corresponding to tap operation) occurs and the number-of-times determining unit 89 determines that the number of times of changes of acceleration corresponding to the shock, that is, the number of times of taps reaches the predetermined number of times (five times), the mode setting unit 90 switches the present operation mode to a power saving mode M5.

The power saving mode M5 is an operation mode for disabling a predetermined function, suppressing power consumption of the measuring device 1, and reducing power consumption. In the power saving mode M5 in this embodiment, the communication function of the communication unit 6, the detection function of the biological-information detecting unit 41, and the detection function of a motion of the measuring device 1 by the acceleration detecting unit 42 are disabled. Therefore, in the power saving mode M5, although the first determination processing by the operation determining unit 86 is not executed, the second determination processing is executed.

That is, in the measuring device 1 according to this embodiment, when the standby mode M1 is set by the mode setting unit 90, if the acceleration detecting unit 42 detects a certain acceleration change (a change in acceleration fixed for a predetermined times), the executing unit 92 executes processing corresponding to the trial mode M2. If the acceleration detecting unit 42 detects another acceleration change (an acceleration change corresponding to continuous five times of tap operation) different from the acceleration change, the executing unit 92 executes processing corresponding to the power saving mode M5 as predetermined processing.

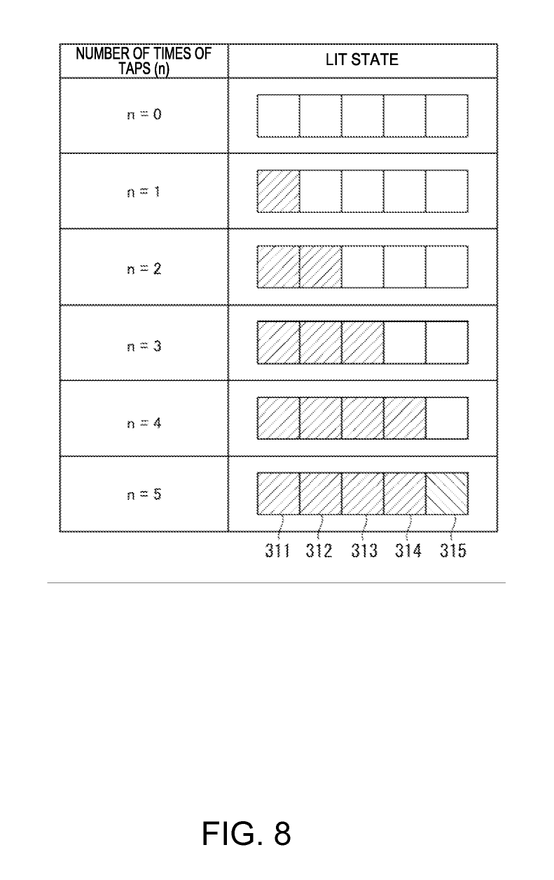

FIG. 8 is a diagram showing a lit state of the display unit 31 corresponding to the number of times of taps.

Note that the number of times of taps counted by the counting unit 87 is notified to the user according to the lit/extinguished states of the LEDs 311 to 315 controlled by the notification control unit 82. Specifically, when the number of times of taps change from “0” to “1”, as shown in FIG. 8, only the LED 311 is lit from a state in which all of the LEDs 311 to 315 are extinguished (a state in which the number of times of taps is “0”). When the number of times of taps changes to “2”, the LEDs 311 and 312 are lit and the LEDs 313 to 315 are extinguished. Thereafter, every time the number of times of taps is incremented, the LEDs 313 to 315 are sequentially lit.

Note that, as explained above, when the predetermined time (e.g., three minutes) elapses after the operation determining unit 86 determines that the tap operation is performed last, the number-of-times initializing unit 88 initializes the number of times of taps counted by the counting unit 87. According to the initialization of the number of times of taps, the notification control unit 82 extinguishes the LEDs 313 to 315.

When the number of times of taps reaches the predetermined number of times (five times) and the operation mode is switched to the power saving mode M5, the number-of-times initializing unit 88 also initializes the number of times of taps.

When the operation mode is the measurement mode M4, even when the predetermined number of times of the tap operation is performed, the regulating unit 91 regulates a change from the measurement mode M4 to the power saving mode M5. As the regulation of the change of the operation mode by the regulating unit 91, the counting of the number of times of taps by the counting unit 87 may be regulated or the mode change itself from the measurement mode M4 to the power saving mode M5 by the mode setting unit 90 may be regulated.

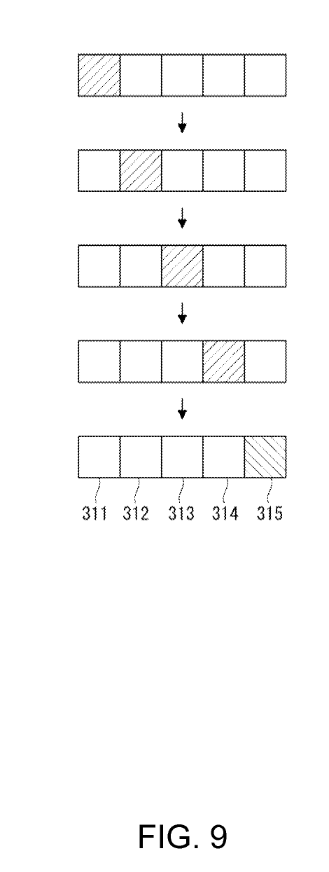

FIG. 9 is a diagram showing a display state by the display unit 31 during the transition to the power saving mode M5.

During the transition to the power saving mode M5, the notification control unit 82 turns on off the LEDs 311 to 315 in order from the LED 311 as shown in FIG. 9. That is, after the display of the number of times of taps is performed, first, all of the LEDs 311 to 315 are extinguished. Then, the LED to be lit is sequentially switched to the LEDs 311 to 315. After the LED 315 lit last is extinguished, the operation mode is switched to the power saving mode M5. According to such display, the user is notified that the operation mode is transitioning to the power saving mode M5 and that the operation mode is switched to the power saving mode M5.

### Explanation of a Start Mode.

When the operation mode is the power saving mode M5, if tap operation same as the tap operation during the change to the power saving mode M5 is performed and the number-of-times determining unit 89 determines that the number of times of taps reaches the predetermined number of times, as shown in FIG. 6, the mode setting unit 90 switches the present operation mode to a start mode M6. When the tap operation is performed in the power saving mode M5, the notification control unit 82 also executes the display (the lighting of the LEDs 311 to 315) corresponding to the number of times of taps.

The start mode M6 is an operation mode in which the executing unit 92 executes start processing for starting the measuring device 1. Note that a progress status of the start processing is notified by lighting of the LEDs 311 and 315 same as the lighting shown in FIG. 9. However, since a processing amount in switching the operation mode to the start mode M6 is larger than a processing amount in switching the operation mode to the power saving mode M5, the lighting transition of the LEDs 311 to 315 progresses slower than the lighting transition in the switching of the operation mode to the power saving mode M5.

When the start processing is completed, the mode setting unit 90 switches the operation mode to the standby mode M1.

### Explanation of a Charging Mode.

On the other hand, when the operation mode is the modes M1 to M5, if the power-feeding-state determining unit 85 detects power supply from the cradle CR and determines that the measuring device 1 and the cradle CR are connected, the mode setting unit 90 switches the present operation mode to a charging mode M7.

The charging mode M7 is an operation mode in which the charging unit 5 charges the secondary battery with supplied power from the cradle CR. In the charging mode M7 in this embodiment, the detection function of the biological-information detecting unit 41 and the detection function of the movement of the measuring device 1 by the acceleration detecting unit 42 are disabled. That is, in the charging mode M7, although the first determination processing by the operation determining unit 86 is not executed, the second determination processing is executed. On the other hand, in the charging mode M7, the communication function of the communication unit 6 is effective. Therefore, the charging mode M7 is equivalent to the communication mode according to the invention in which communication with the external apparatus is possible.

When the operation mode is the charging mode M7, the notification control unit 82 notifies a charged state of the secondary battery with a lit state of the LEDs 311 to 315 under the control by the executing unit 92.

Although not shown in the figure, when the secondary battery is being charged, the LEDs 311 to 314 are extinguished and the LED 315 is lit. When the charging of the secondary battery ends, the LED 311 is lit and the LEDs 312 to 315 are extinguished. On the other hand, when the secondary battery cannot be charged because of some deficiency, the LEDs 311 to 314 are extinguished and only the LED 315 is blinked.

### Explanation of a Communication Release Mode.

When the operation mode is the charging mode M7, if tap operation same as the tap operation during the switching from the standby mode M1 to the power saving mode M5 is performed and the number-of-times determining unit 89 determines that the number of times of taps reaches the predetermined number of times, the mode setting unit 90 switches the present operation mode to a communication release mode M8. Note that, when the tap operation is performed in the charging mode M7, the notification control unit 82 also executes the display (the lighting of the LEDs 311 to 315) corresponding to the number of times of taps.

The communication release mode M8 is an operation mode for erasing the connection information stored in the storing unit 7 and releasing the connection to the external apparatus associated with the measuring device 1. In the communication release mode M8 in this embodiment, the communication function of the communication unit 6 and the detection function of the detecting units 41 and 42 are disabled.

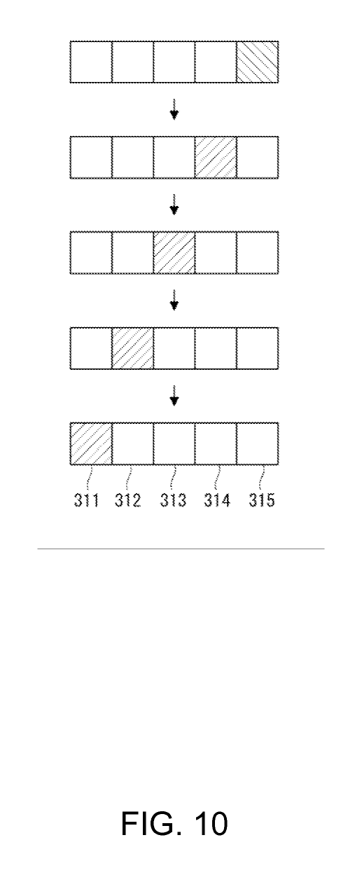

FIG. 10 is a diagram showing a display state by the display unit 31 in the communication release mode M8.

When the operation mode is the communication release mode M8, the notification control unit 82 notifies a progress status of processing in the communication release mode M8 with a lit state of the LEDs 311 to 315 under the control by the executing unit 92.

Specifically, as shown in FIG. 10, after extinguishing the LEDs 311 to 315, the notification control unit 82 switches the LED to be lit in order from the LED 315 to the LED 311. According to such display, the user is notified that deletion of the connection information and release of the communication connection are performed.

When processing executed in the communication release mode M8 ends, the mode setting unit 90 switches the operation mode to the charging mode.

### Update Mode.

When the operation mode is the charging mode M7 and the measuring device 1 is connected to the external apparatus via the communication unit 6, if an update program is received from the external apparatus, as shown in FIG. 6, the mode setting unit 90 switches the present operation mode to an update mode M9.

The update mode M9 is an operation mode in which the executing unit 92 updates, according to the received update program, the computer program stored in the storing unit 7. In the update mode M9 in this embodiment, the communication function of the communication unit 6 and the detection function of the detecting units 41 and 42 are disabled.

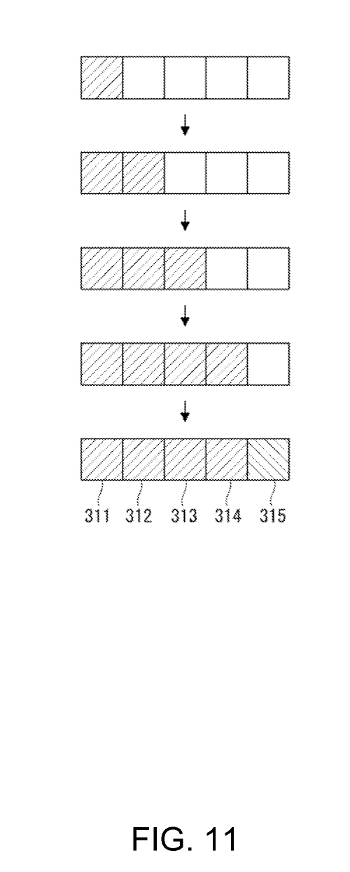

FIG. 11 is a diagram showing a display state by the display unit 31 in the update mode M9.

When the operation mode is switched to the update mode M9, the notification control unit 82 notifies a progress status of processing in the update mode M9 with a lit state of the LEDs 311 to 315 under the control by the executing unit 92.

Specifically, as shown in FIG. 11, after extinguishing the LEDs 311 to 315, the notification control unit 82 lights, according to the progress status, the LEDs 311 to 315 in order from the LED 311 to the LED 315. According to such display, the user is notified that update processing for the computer program (firmware) is performed.

When the update processing for the computer program in the update mode M9 ends, the mode setting unit 90 switches the operation mode to the start mode M6 and switches the operation mode to the standby mode M1 through the start mode M6.

### Reset Mode.

On the other hand, when the operation mode is the charging mode M7, if the measuring device 1 is detached from the cradle CR (if the power supply from the cradle CR is stopped), as shown in FIG. 6, the mode setting unit 90 switches the present operation mode to a reset mode M10.

The reset mode M10 is an operation mode in which the executing unit 92 executes reset processing (system reset) for the entire measuring device 1.

When the reset processing is completed, the mode setting unit 90 switches the operation mode to the start mode M6. When the start processing in the start mode M6 is completed, the operation mode is switched to the standby mode M1.

### Other Operation Modes.

The mode setting unit 90 switches the operation mode not only to the modes M1 to M10 but also to other modes according to operation for the measuring device 1 and a state of the measuring device 1.

For example, when a battery voltage of the secondary battery is equal to or lower than a first threshold, the mode setting unit 90 switches the operation mode to a shutdown mode. The shutdown mode is an operation mode in which the executing unit 92 stops the functions of the communication unit 6 and the detecting units 41 and 42. In the shutdown mode in this embodiment, the notification control unit 82 extinguishes the LEDs 311 to 314 and blinks the LED 315 for a predetermined time (e.g., thirty seconds) under the control by the executing unit 92.

When the battery voltage of the secondary battery is lower than a second threshold lower than the first threshold, the mode setting unit 90 switches the operation mode to a battery protection mode. In the battery protection mode in this embodiment, in addition to the processing performed in the shutdown mode, all of the LEDs 311 to 315 are extinguished. Besides, the secondary battery is protected and set in a state same as a factory shipment state.

Note that, when the operation mode is any one of the shutdown mode or the battery protection mode, if the measuring device 1 is connected to the cradle CR and power supply from the cradle CR is detected, the mode setting unit 90 switches the operation mode to the charging mode.

On the other hand, when the operation mode is the measuring mode M4, if a communication request is received from an external apparatus such as a multifunction cellular phone or a PC (Personal Computer), the mode setting unit 90 switches the operation mode to a connection mode. In the connection mode in this embodiment, the communication control unit 83 establishes communication connection to the external apparatus under the control by the executing unit 92. In this case, the executing unit 92 causes the storing unit 7 to store connection information necessary for the communication connection to the external apparatus. Besides, the notification control unit 82 causes, according to a progress status of establishing processing for the communication connection, the display unit 31 to execute the display (the lighting of the LEDs 311 to 315) shown in FIG. 9. When such processing is completed, the mode setting unit 90 switches the operation mode to the measurement mode M4, which is the mode before the switching.

Further, when the operation mode is the connection mode, if an event occurrence notice is received from the external apparatus to which the communication connection is established, the mode setting unit 90 switches the operation mode to an event notification mode. In the event notification mode in this embodiment, the notification control unit 82 causes the display unit 31 to carry out the display (the lighting of the LEDs 311 to 315) shown in FIG. 9 three times under the control by the executing unit 92 to thereby notify the user that some event occurs.

Examples of such an event include an event in which an electronic mail is received by the external apparatus, an event in which the present time reaches notification time (alarm time) set in advance, and an event in which the multifunction cellular phone, which is the external apparatus, receives a telephone call.

After the display processing in the event notification mode is performed, the mode setting unit 90 switches the operation mode to the communication mode, which is the mode before the switching.

### Mode Setting Processing.

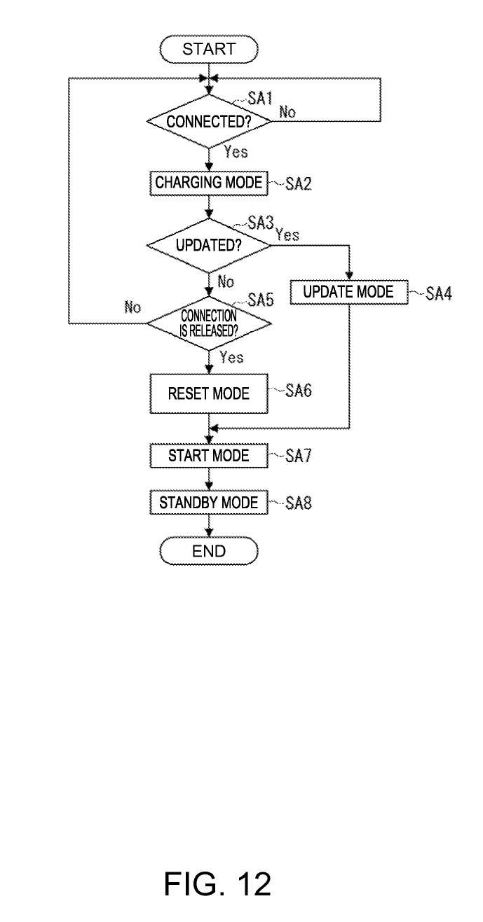

FIGS. 12 to 14 are flowcharts for explaining mode setting processing executed by the control unit 8.

The control unit 8 executes first mode setting processing to third mode setting processing shown in FIGS. 12 to 14, whereby the mode setting unit 90 sets the operation mode.

The respective kinds of mode setting processing are explained below.

### First Mode Setting Processing.

When the present operation mode is any one of the modes M1 to M4, the control unit 8 executes the first mode setting processing shown in FIG. 12 and sets the operation mode.

In the first mode setting processing, first, the control unit 8 determines on the basis of a determination result by the power-feeding-state determining unit 85 whether the measuring device 1 is connected to the cradle CR (step SA1). When determining that the measuring device 1 is not connected to the cradle CR, the control unit 8 returns the processing to step SA1.

On the other hand, when the control unit 8 determines in determination processing in step SA1 that the measuring device 1 is connected to the cradle CR, the mode setting unit 90 switches the present operation mode to the charging mode M7 (step SA2).

Thereafter, the control unit 8 determines whether an update program is received from an external apparatus (step SA3).

When the control unit 8 determines in determination processing in step SA3 that the update program is received, the mode setting unit 90 switches the present mode to the update mode M9 (step SA4). When the executing unit 92 executes processing in the update mode M9, the control unit 8 shifts the processing to step SA7.

When determining in the determination processing in step SA3 that the update program is not received, the control unit 8 determines on the basis of a determination result by the power-feeding-state determining unit 85 whether the measuring device 1 is detached from the cradle CR (step SA5). When determining that the measuring device 1 is not detached from the cradle CR, the control unit 8 returns the processing to step SA1.

On the other hand, when the control unit 8 determines in determination processing in step SA5 that the measuring device 1 is detached from the cradle CR, the mode setting unit 90 switches the present operation mode to the reset mode M10 (step SA6). When reset processing by the executing unit 92 in step SA6 is completed, the control unit 8 shifts the processing to step SA7.

In step SA7, the mode setting unit 90 switches the present operation mode to the start mode M6 (step SA7).

When start processing in the start mode M6 is completed, the mode setting unit 90 further switches the present operation mode to the standby mode M1 (step SA8).

When the present operation mode is switched to the standby mode M1 in this way, the first mode setting processing is executed again.

### Second Mode Setting Processing.

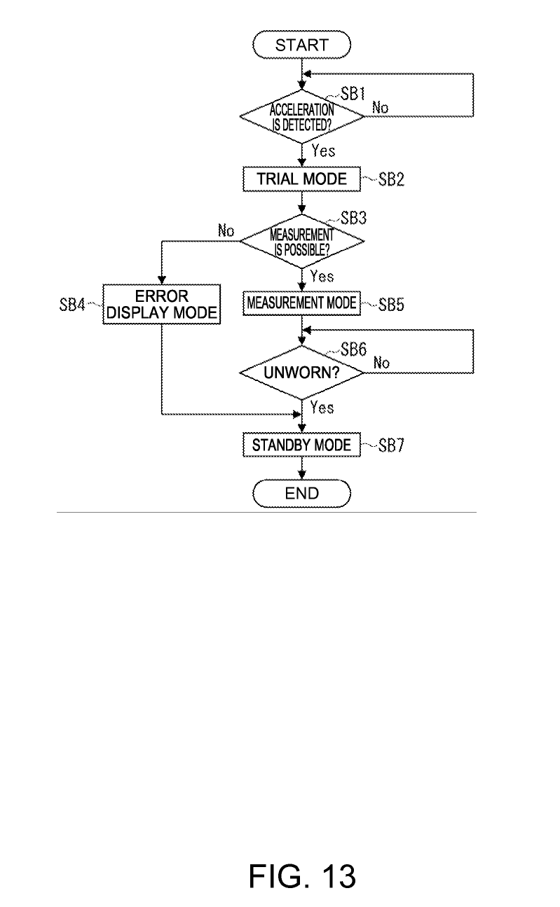

When the present operation mode is the standby mode M1, the control unit 8 executes the second mode setting processing shown in FIG. 13 together with the first mode setting processing and sets the operation mode.

In the second mode setting processing, first, the control unit 8 determines on the basis of a detection result by the operation determining unit 86 whether an acceleration value changes (step SB1). When determining that there is no change in the acceleration value, the control unit 8 returns the processing to step SB1.

On the other hand, when the control unit 8 determines that a change occurs in the acceleration value, the mode setting unit 90 switches the present operation mode to the trial mode M2 (step SB2).

After step SB2, the measurement-possibility determining unit 84 determines whether biological information is detected, that is, whether measurement of biological information is possible (step SB3). This determination processing is repeatedly executed during the trial period.

When the measurement-possibility determining unit 84 determines that the measurement of the biological information is not possible, the mode setting unit 90 switches the present operation mode to the error display mode M3 (step SB4). Thereafter, the control unit 8 shifts the processing to step SB7.

On the other hand, when the measurement-possibility determining unit 84 determines that the measurement of the biological information is possible, the mode setting unit 90 switches the present operation mode to the measurement mode M4. The executing unit 92 executes an analysis and recording of the biological information (step SB5).

After step SB5, the control unit 8 determines whether the measuring device 1 is unworn for a predetermined time (three minutes) (step SB6). While the measuring device 1 is worn, the determination processing in step SB6 is repeatedly executed.

On the other hand, when determining that the measuring device 1 is unworn for the predetermined time, the control unit 8 shifts the processing to step SB7.

In step SB7, the mode setting unit 90 switches the present operation mode to the standby mode M1 (step SB7).

When the present operation mode is switched to the standby mode M1 in this way, like the first mode setting processing, the second setting processing is executed again.

### Third Mode Setting Processing.

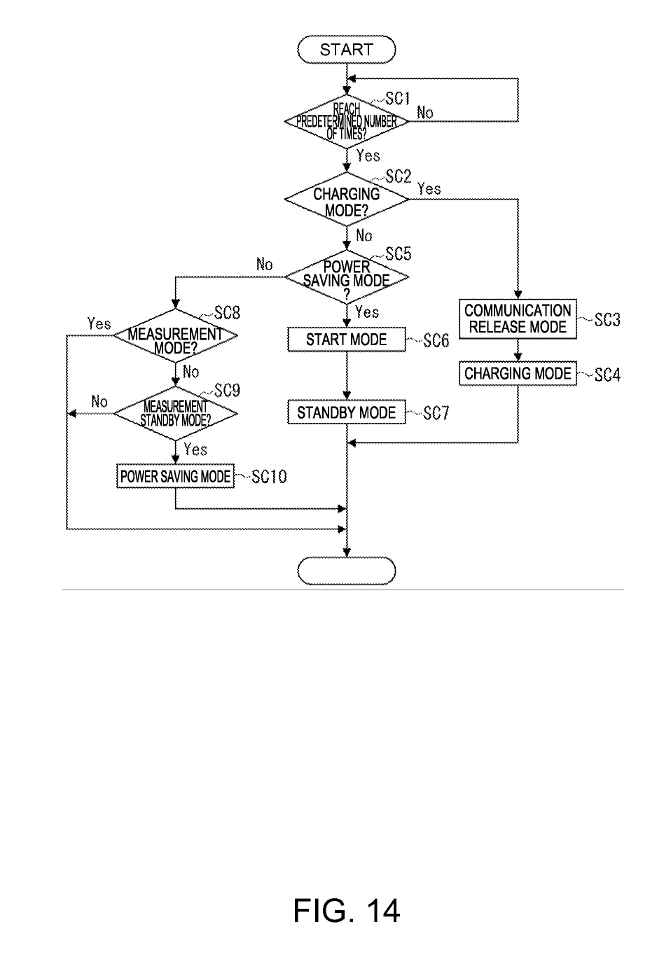

When the operation determining unit 86 determines that the tap operation is performed, the control unit 8 executes the third mode setting processing shown in FIG. 14. The third mode setting processing is executed when the present operation mode is an operation mode different from the shutdown mode and the battery protection mode.

In the third mode setting processing, first, the number-of-times determining unit 89 determines whether the number of times of taps reaches the predetermined number of times (step SC1). When the number-of-times determining unit 89 determines that the number of times of taps does not reach the predetermined number of times, the control unit 8 returns the processing to step SC1.

On the other hand, when the number-of-times determining unit 89 determines that the number of times of taps reaches the predetermined number of times, the mode setting unit 90 determines that the present operation mode is the charging mode M7 (step SC2).

When the mode setting unit 90 determines in the determination processing in step SC2 that the present operation mode is the charging mode M7, the mode setting unit 90 switches the present operation mode to the communication release mode M8 (step SC3).

When the processing in the communication release mode M8 is completed, the mode setting unit 90 switches the operation mode to the charging mode M7 (step SC4).

After step SC4, the control unit 8 ends the third mode setting processing.

When determining in the determination processing in step SC2 that the present operation mode is not the charging mode M7, the mode setting unit 90 determines whether the operation mode is the power saving mode M5 (step SC5).

When determining in determination processing in step SC5 that the present operation mode is the power saving mode M5, the mode setting unit 90 switches the operation mode to the start mode M6 (step SC6).

When start processing in the start mode M6 is completed, the mode setting unit 90 switches the operation mode to the standby mode M1 (step SC7).

After step SC7, the control unit 8 ends the third mode setting processing.

When determining in the determination processing in step SC5 that the present operation mode is not the power saving mode M5, the mode setting unit 90 determines whether the operation mode is the measurement mode M4 (step SC8).

When the mode setting unit 90 determines that the present operation mode is the measurement mode M4, the regulating unit 91 regulates the change from the measurement mode M4 to the power saving mode M5. Therefore, the control unit 8 ends the third mode setting processing.

On the other hand, when determining that the present operation mode is not the measurement mode M4, the mode setting unit 90 determines whether the operation mode is a measurement standby mode, which is an operation mode that can be changed to the power saving mode M5 (step SC9). Specifically, in step SC9, the mode setting unit 90 determines whether the present operation mode is any one of the modes M1 to M3.

When determining that the present operation mode is the measurement standby mode, the mode setting unit 90 switches the present operation mode to the power saving mode (step SC10). Thereafter, the control unit 8 ends the third mode setting processing.

On the other hand, when determining that the present operation mode is not the measurement standby mode, the mode setting unit 90 does not change the operation mode. The control unit 8 ends the third mode setting processing.

In this way, the operation mode of the measuring device 1 is set.

### Effects of the Embodiment

With the measuring device 1 according to the embodiment explained above, effects explained below are obtained.

The mode setting unit 90 switches, on the basis of a change pattern of an acceleration value detected by the acceleration detecting unit 42, the present operation mode from the standby mode M1 to any one of the trial mode M2 and the power saving mode M5. The executing unit 92 executes processing corresponding to the set operation mode. That is, when it is detected that an acceleration value changes after being fixed for a predetermined time, the mode setting unit 90 switches the present operation mode to the trial mode M2. When a change in the acceleration value corresponding to a continuous shock by the predetermined number of times of the tap operation is detected, the mode setting unit 90 switches the present operation mode to the power saving mode M5. Consequently, it is possible to easily switch the operation mode to the trial mode M2 by moving the measuring device 1, the operation mode of which is the standby mode M1. It is possible to easily switch the operation mode to the power saving mode M5 by performing the predetermined number of times of the tap operation on the measuring device 1. It is possible to cause the executing unit 92 to execute the processing corresponding to the modes M2 and M5. Therefore, the processing to be executed can be changed according to the change pattern of the acceleration without depending on input operation of a button or the like. Therefore, it is possible to attain a reduction in the size of the measuring device 1 and reduce a burden on the user who wears the measuring device 1.

Even when the same operation (e.g., continuous tap operation) is carried out when different operation modes are set, it is possible to execute processing corresponding to the present operation mode and the detected change pattern of the acceleration. Further, since the processing can be executed without providing a machine mechanism such as a button exposed to the outside, it is possible to secure a waterproof property without providing a special component. Consequently, it is also possible to reduce costs.

When the present operation mode is the standby mode M1, if the acceleration value changes, the mode setting unit 90 switches the present operation mode to the trial mode M2. Consequently, when the measuring device 1 is lifted to be worn on the human body from a state in which the measuring device 1 is left untouched on a table or the like, since the operation mode is switched to the trial mode M2, it is possible to surely carry out detection of biological information when the measuring device 1 is worn. Therefore, it is possible to suppress occurrence of a measurement omission of the biological information.

When the present operation mode is the standby mode M1, if a change pattern of an acceleration value corresponding to a continuous shock by the tap operation is detected, the mode setting unit 90 switches the present operation mode to the power saving mode M5. Consequently, it is possible to switch the operation mode to the power saving mode M5 with simple operation. Besides, it is possible to reduce power consumption of the measuring device 1. Therefore, it is unnecessary to frequency charge the secondary battery. It is possible to improve convenience of the measuring device 1.

When the present operation mode is the measurement mode M4, the regulating unit 91 regulates switching of the operation mode to the power saving mode M5 by the mode setting unit 90. Consequently, it is possible to suppress, in a state in which biological information is measured, the operation mode from being switched to the power saving mode to suspend the measurement of the biological information. Therefore, it is possible to appropriately measure the biological information. When another kind of processing, for example, a function of causing the display unit to display a state of a pulse rate is allocated to single tap operation, clearly different operation of “tapping a plurality of times” is necessary. Therefore, it is possible to prevent the processing from being confused with processing executed by continuous tap operation.

When the present operation mode is the power saving mode M5, if a change pattern of an acceleration value corresponding to a continuous shock by the tap operation is detected, the mode setting unit 90 releases the power saving mode M5 and switches the present operation mode to the start mode M6. Consequently, it is possible to easily switch the operation mode from the power saving mode M5 to the standby mode M1 through an operation mode in which the measuring device 1 can be used, that is, the start mode M6. Therefore, it is possible to improve operability of the measuring device 1. Besides, it is possible to easily carry out detection and measurement of biological information and suppress occurrence of a measurement omission of the biological information.

The measuring device 1 includes the communication unit 6 that communicates with the external apparatus. When the present operation mode is the power saving mode M5, if electric power is supplied to the measuring device 1 from the cradle CR, the mode setting unit 90 switches the present operation mode to the charging mode M7, which is a communication mode in which the measuring device 1 can communicate with the external apparatus. Consequently, it is possible to carry out the communication with the external apparatus, in which power consumption is relatively large, using the supplied electric power. Therefore, it is possible to communicate with the external apparatus without communication connection being interrupted by battery exhaustion.

When electric power is supplied to the measuring device 1 (i.e., when the operation mode is the charging mode), if a change pattern of an acceleration value corresponding to a continuous shock of the tap operation is detected, the mode setting unit 90 switches the present operation mode to the communication release mode M8. In the communication release mode M8, the executing unit 92 deletes the connection information to the external apparatus stored in the storing unit 7. Consequently, when the power supply from the cradle CR is performed, since communication with the external apparatus associated with the measuring device 1 can be released, it is possible to perform communication connection to different external apparatuses. Therefore, since a connection destination of the measuring device 1 can be changed, it is possible to improve convenience of the measuring device 1.

When the power supply to the measuring device 1 is stopped, the mode setting unit 90 switches the present operation mode to the reset mode M10 and causes the executing unit 92 to reset the measuring device 1. Thereafter, the mode setting unit 90 switches the present operation mode to the start mode M6 and then switches the present operation mode to the standby mode M1. Consequently, since the measuring device 1 can be reset at timing when power supply from the outside is stopped, it is possible to execute system reset without the user being aware of the system rest. Besides, it is possible to cause the measuring device 1 to stably operate. Start processing after the system reset is executed requires relatively large electric power. However, since the secondary battery is charged by the supplied electric power, it is possible to suppress the start processing from being stopped by battery exhaustion.

### Modifications of the Embodiment

The invention is not limited to the embodiment. Modifications, improvements, and the like within a range in which the object of the invention can be attained are included in the invention.

In the embodiment, when the present operation mode is the standby mode M1, if an acceleration value changes (i.e., in a change pattern in which an acceleration value fixed for a predetermined period changes), the mode setting unit 90 switches the operation mode to the trial mode M2. When the present operation mode is any one of the standby mode M1, the trial mode M2, and the error display mode M3, if a change pattern of a detected acceleration value is a change pattern of an acceleration value corresponding to a continuous shock by the tap operation, the mode setting unit 90 switches the operation mode to the power saving mode M5. However, the invention is not limited to this. The operation mode before the switching by the mode setting unit 90 is not limited to the standby mode M1 and may be other operation modes.

The operation mode after the switching by the mode setting unit 90 is not limited to the trial mode M2 and the power saving mode M5 and may be other operation modes. Processing different from the processing executed in the modes M2 and M5 may be executed. Further, the mode setting unit 90 may switch the operation mode to other operation modes according to other change patterns of acceleration values. That is, the processing executed when the predetermined acceleration change is detected does not have to be the processing corresponding to the modes M2, M5, and M8. The acceleration change does not have to be the pattern corresponding to the change in the acceleration after being fixed for the predetermined period or the pattern corresponding to the continuous tap operation.

In addition, the change pattern of the acceleration in switching to the power saving mode M5 is not limited to the change pattern of the acceleration based on the shock corresponding to the five times of the tap operation and may be other change patterns. The number of times of the tap operation for switching the operation mode may be less than five times or equal to or more than six times. For example, the operation mode to be switched and the processing to be executed may be classified according to the number of times of taps per unit time and a cycle of the tap operation.

Not that an upper limit of the number of times of taps in switching the operation mode is preferably ten times. One reason for this is that, since an area of an arrangement part of a display unit is limited in a wearable device, if the upper limit of the number of times of taps for executing the predetermined processing exceeds ten times, it is difficult to grasp whether the number of times of taps to be displayed (a counted number of times of taps) is enough for executing the processing.

Specifically, in the measuring device 1, the number of LEDs configuring the display unit 31 and indicating the number of times of taps by being lit and extinguished is set to five. It is possible to display taps ten times at most by reciprocatingly lighting the five LEDs 311 to 315 arrayed along the longitudinal direction of the front surface section 21A. However, if it is attempted to display taps more than eleven times, it is necessary to perform the reciprocating lighting twice or more. The user less easily grasps the number of times of taps. On the other hand, it is conceivable to increase the arrangement part and increase the display unit in size (increase the number of LEDs). However, in this case, the wearable device is increased in size. Therefore, when a balance between a reduction in the size of the wearable device and easiness in grasping the number of times of taps is taken into account, the upper limit of the number of times of taps for executing the switching of the operation mode is preferably ten times.

Note that the processing to be executed may be varied according to the number of times of taps. For example, when the number of times of continuous taps reaches five times, the operation mode may be switched to the power saving mode M5 or the communication release mode M8 according to a state of the measuring device 1. When the number of times of continuous taps reaches ten times, the operation mode may be switched to the reset mode M10.

In the embodiment, when the present operation mode is the measurement mode M4, the regulating unit 91 regulates a change to the power saving mode M5 by the mode setting unit 90. However, the invention is not limited to this. That is, the regulation of the change of the operation mode by the regulating unit 91 does not have to be carried out. On the other hand, the regulating unit 91 may regulate the switching to another operation mode by the mode setting unit 90 under a certain condition.

In the embodiment, when the present operation mode is the power saving mode M5, if the change pattern of the acceleration value corresponding to continuous five times of shocks by the tap operation is detected, the mode setting unit 90 releases the power saving mode M5 and switches the operation mode to the start mode M6. However, the invention is not limited to this. That is, the operation for switching the present operation mode from the power saving mode M5 to the start mode M6 may be other kinds of operation.

In the embodiment, when the power supply from the outside is detected, the mode setting unit 90 switches the present operation mode to the charging mode M7. The communication function of the communication unit 6 is enabled. However, the invention is not limited to this. That is, in the charging mode M7, the secondary battery only has to be chargeable by the charging unit 5. The communication function of the communication unit 6 may be either effective or ineffective. Further, the measuring device 1 includes the secondary battery. However, the invention is not limited to this. The measuring device 1 may include a primary battery that supplies driving power for the measuring device 1.

In the embodiment, when the present operation mode is the charging mode M7, if a change pattern of an acceleration value corresponding to a continuous shock by five times of the tap operation is detected, the present operation mode is switched to the communication release mode M8. The connection information stored in the storing unit 7 is deleted. However, the invention is not limited to this. For example, the connection information may be deleted by another kind of operation. The operation mode before the switching to the communication release mode M8 is not limited to the charging mode M7 and may be other operation modes.

The trial time and the predetermined times can be changed as appropriate.

In the embodiment, when the present operation mode is the charging mode M7, if the measuring device 1 is detached from the cradle CR and the power supply from the cradle CR is stopped, the mode setting unit 90 switches the present operation mode to the reset mode M10. However, the invention is not limited to this. The operation mode may be switched to the standby mode M1 and the other operation modes not through the reset mode M10 for resetting the measuring device 1 and the start mode M6.

In the embodiment, the acceleration detecting unit 42 detects an acceleration value. However, the invention is not limited to this. That is, the acceleration detecting unit 42 may be unable to detect an acceleration value if the acceleration detecting unit 42 can detect a change in acceleration.

In the embodiment, the measuring device 1 detects a pulse wave of the user as biological information and analyzes the pulse wave to measure a pulse. However, the invention is not limited to this. That is, the measuring device according to the invention may be a measuring device that detects and measures other kinds of biological information such as a body temperature, a blood pressure, an electrocardiogram, and a brain wave.

The measuring device 1 is configured as the wearable device of the wristwatch type worn on the wrist of the user. However, the invention is not limited to this. That is, the part of the human body on which the measuring device 1 is worn is not limited to the wrist and may be other parts such as an ankle.

In the embodiment, the measuring device 1 includes the five LEDs 311 and 315. However, the invention is not limited to this. That is, the number of LEDs may be three or may be seven. Further, if the number of times of taps and the number of LEDs are equal or when one is an integer times as large as the other, a tap operation state is intuitively easily grasped. Therefore, it is possible to provide a user-friendly user interface.

In the embodiment, the enabling and the disabling of the communication function of the communication unit 6 are set according to the operation modes. The invention is not limited to the configuration in the embodiment. That is, when the operation mode other than the power saving mode M5, the communication function of which is disabled, is set, the communication function is enabled. When a communication request is received from the external apparatus, the processing in the set present operation mode and the processing in the connection mode may be respectively executed.

In the embodiment, when the operation mode is the charging mode M7 and the measuring device 1 is connected to the external apparatus via the communication unit 6, if the update program is received from the external apparatus, the operation mode is switched to the update mode M9. However, the invention is not limited to this. For example, the operation mode can be switched to the update mode M9 if the measuring device 1 is connected to the cradle CR. In this case, the connection to the cradle CR may be detected using a physical switch, a photo sensor, or the like that comes into contact with the cradle CR and projects and retracts.

What is claimed is:

1. A biological information measuring device comprising:
a biological-information detecting unit configured to detect biological information of a human body on which the biological information measuring device is worn;
an acceleration detecting unit configured to detect acceleration;
a mode setting unit configured to set, among a plurality of operation modes set on the basis of a change in the detected acceleration, an operation mode corresponding to a change pattern of the acceleration; and
an executing unit configured to execute processing corresponding to the set operation mode.

2. The biological information measuring device according to claim 1, wherein, when the operation mode is a standby mode for standing by for detection of the biological information by the biological-information detecting unit, if the acceleration changes, the mode setting unit switches the operation mode to a detection mode for detecting the biological information.

3. The biological information measuring device according to claim 1, wherein, when the operation mode is a standby mode for standing by for detection of the biological information by the biological-information detecting unit, if a change in the acceleration corresponding to a continuous shock is detected, the mode setting unit switches the operation mode to a power saving mode for reducing power consumption to be smaller than power consumption in the standby mode.

4. The biological information measuring device according to claim 3, further comprising a regulating unit configured to regulate, when the operation mode is a measurement mode for measuring the biological information, setting of the power saving mode by the mode setting unit.

5. The biological information measuring device according to claim 3, wherein, when the operation mode is the power saving mode, if the change in the acceleration corresponding to the continuous shock is detected, the mode setting unit releases the power saving mode.

6. The biological information measuring device according to claim 3, further comprising a communication unit configured to communicate with an external apparatus, wherein
when the operation mode is the power saving mode, if electric power is supplied from an outside, the mode setting unit switches the operation mode to a communication mode in which the measuring device can communicate with the external apparatus via the communication unit.

7. The biological information measuring device according to claim 6, wherein, when the electric power is supplied from the outside, if the change in the acceleration corresponding to the continuous shock is detected, the mode setting unit switches the operation mode to a communication release mode for releasing the communication with the external apparatus, and the executing unit deletes connection information with the external apparatus stored in advance.

8. The biological information measuring device according to claim 6, wherein, when the power supply from the outside is stopped, after switching the operation mode to a reset mode and causing the executing unit to execute reset processing, the mode setting unit switches the operation mode to a start mode for causing the executing unit to execute start processing.

9. A control method for a biological information measuring device worn on a human body to measure biological information, the control method comprising:
detecting acceleration;
setting, among a plurality of operation modes set on the basis of a change in the detected acceleration, an operation mode corresponding to a change pattern of the acceleration; and
executing processing corresponding to the set operation mode.

10. A biological information measuring device comprising:
a biological-information detecting unit configured to detect biological information of a human body on which the biological information measuring device is worn;
an acceleration detecting unit configured to detect acceleration;
a mode setting unit configured to set an operation mode on the basis of a change in the detected acceleration; and
an executing unit configured to execute predetermined processing when a predetermined acceleration change is detected by the acceleration detecting unit in the set operation mode.
